(19) United States
(12) Patent Application Publication
(10) Pub. No.: US 2020/0190737 A1
(43) Pub. Date: Jun. 18, 2020

(54) CONTINUOUS PILOT SCALE HYDROTHERMAL HORIZONTAL REACTOR DESIGN FOR MAKING INDUSTRIAL BY-PRODUCTS

(71) Applicant: Tyton Biosciences, LLC, Danville, VA (US)

(72) Inventors: Florin G. BARLA, Danville, VA (US); Iulian BOBE, Danville, VA (US); Hsun-Cheng SU, Chapel Hill, NC (US)

(21) Appl. No.: 16/424,869

(22) Filed: May 29, 2019

Related U.S. Application Data
(63) Continuation of application No. PCT/US17/65334, filed on Dec. 8, 2017.
(60) Provisional application No. 62/431,891, filed on Dec. 9, 2016.

(51) Int. Cl. D21C 7/04; D21C 7/10; D21B 1/06; D21C 3/02; D21C 5/02; D21H 11/14; D21H 11/12; C07H 1/08; B01F 7/00; B01F 7/04

(52) U.S. Cl. CPC D21C 7/04; D21C 7/10; D21B 1/063; D21C 3/02; D21C 5/02; D21H 11/14; B01F 2215/0078; C07H 1/08; B01F 7/00116; B01F 7/00175; B01F 7/00208; B01F 7/041; D21H 11/12

(57) ABSTRACT

The presently disclosed subject matter relates to a horizontal reactor system that can be used for processing various plant material biomass to produce fermentable carbohydrates, paper and pulp products, or both. Particularly, the disclosed system comprises a horizontal reactor vessel comprising a body with first and second ends. The reaction vessel is intended to encompass and retain a reaction medium (i.e., biomass slurry) that is to undergo a biological and/or biochemical reaction. The vessel comprises an internal agitator that functions to mix the contents of the reactor. The agitator is powered by a drive, which can be a motor, positioned outside the reaction vessel.

### CROSS REFERENCE TO RELATED APPLICATIONS

This application is a continuation of PCT patent application no. PCT/US17/65334 filed on Dec. 8, 2017, which claims priority to and the benefit of U.S. Provisional Patent Application No. 62/431,891, filed Dec. 9, 2016, which is incorporated by reference herein in its entirety.

### TECHNICAL FIELD

The presently disclosed subject matter relates to a horizontal reactor system and to methods of making and using the disclosed system. Particularly, the presently disclosed subject matter is directed to a continuous pilot scale hydrothermal horizontal reactor design for making pulp and sugars using agricultural waste and other lignocellulosic industrial by-products.

### BACKGROUND

Vertical reactor systems have conventionally been used to process biomass. Particularly, vertical reactor systems are simple in design and relatively inexpensive, both of which are advantageous at larger scales. However, working with biomass and lignocellulosic material in a vertical flow reactor, the insoluble materials generated from the subcritical water hydrolysis process exhibit a higher sedimentation rate. As a result, due to the problem of inefficient mixing, existing vertical reactor systems cannot effectively process biomass. It would therefore be beneficial to provide an alternative to the vertical reactor system that promotes more effective mixing.

### SUMMARY

In some embodiments, the presently disclosed subject matter is directed to A horizontal bioreactor apparatus comprising a horizontal reaction vessel comprising an interior and first and second ends; a first hub positioned at the first end of the vessel; a second hub positioned at the second end of the vessel; and an agitator positioned within the interior of the vessel. The agitator comprises: a central rotating bore operably connected to the first and second hubs; and one or more arms connected to the central rotating bore and operably connected to the first and second hubs.

In some embodiments, the horizontal reaction vessel is constructed from stainless steel or a polymeric material.

In some embodiments, the central rotating bore is coupled to an external drive device via the first or second hub. In some embodiments, the external drive device is a motor.

In some embodiments, the horizontal reaction vessel is capable of processing about 1-2 liters of biomass slurry per minute.

In some embodiments, the horizontal reaction vessel can accommodate pressures of up to about 1,550 psi.

In some embodiments, the horizontal reaction vessel can accommodate temperatures of up to about 450° F.

In some embodiments, the horizontal reaction vessel r is pressurized through the use of a gas tank. In some embodiments, the gas is nitrogen.

In some embodiments, the arms contact the interior surface of the reactor.

In some embodiments, the apparatus comprises a viewing glass for monitoring progress of the reactions in the vessel.

In some embodiments, the apparatus comprises a sample port.

In some embodiments, the presently disclosed subject matter is directed to a horizontal bioreactor system comprising: a mechanical pre-treatment module; a bioreactor module comprising a horizontal reaction vessel as disclosed herein; and a collection module.

In some embodiments, the mechanical pre-treatment module comprises a hammer mill.

In some embodiments, the system comprises a heating module comprising a heat exchanger. In some embodiments, the heat exchanger comprises metal tube elements housing heated or cooled liquid.

In some embodiments, the presently disclosed subject matter is directed to a method of processing biomass to produce fermentable carbohydrates, paper and pulp products, or both. The method comprises preparing a biomass slurry comprising a biomass to be treated; introducing the biomass slurry to a horizontal bioreactor apparatus as disclosed herein; allowing the horizontal bioreactor apparatus to proceed for a desired amount of time; and collecting end products of fermentable carbohydrates, paper and pulp products, or both.

In some embodiments, the biomass is selected from corn, wheat, soybean, cabbage, sugar beet, sugar cane, greens, tobacco, cotton linter, bamboo, lavender, algae, Artemisia, hemp, lumber, chipped wood, corn stover, potato sacks, coffee sacks, paper cups, and combinations thereof.

In some embodiments, the biomass slurry comprises biomass and a solvent selected from water, NaOH, buffer, and combinations thereof.

In some embodiments, the ratio of biomass to solvent is about 1:7 to 1:20.

### BRIEF DESCRIPTION OF THE DRAWINGS

The previous summary and the following detailed descriptions are to be read in view of the drawings, which illustrate some (but not all) embodiments of the presently disclosed subject matter.

FIG. 1a is a line drawing of one embodiment of the disclosed reactor.

FIG. 1b is a perspective view of one embodiment of a reactor in accordance with the presently disclosed subject matter.

FIG. 2a is a perspective view of one embodiment of an agitator that can be used in accordance with some embodiments of the presently disclosed subject matter.

FIG. 2b is a cutaway view of one embodiment of the agitator of FIG. 2a positioned in a reactor.

FIG. 3 is a line drawing of a system comprising the disclosed reactor in accordance with some embodiments of the presently disclosed subject matter.

FIG. 4 is one embodiment of a heat exchanger that can be used in accordance with the presently disclosed subject matter.

FIGS. 5a and 5b are perspective views of the disclosed system in accordance with some embodiments of the presently disclosed subject matter.

### DETAILED DESCRIPTION

The presently disclosed subject matter is introduced with sufficient details to provide an understanding of one or more particular embodiments of broader inventive subject matters. The descriptions expound upon and exemplify features of those embodiments without limiting the inventive subject matters to the explicitly described embodiments and features. Considerations in view of these descriptions will likely give rise to additional and similar embodiments and features without departing from the scope of the presently disclosed subject matter.

Unless defined otherwise, all technical and scientific terms used herein have the same meaning as commonly understood to one of ordinary skill in the art to which the presently disclosed subject matter pertains. Although any methods, devices, and materials similar or equivalent to those described herein can be used in the practice or testing of the presently disclosed subject matter, representative methods, devices, and materials are now described.

Following long-standing patent law convention, the terms “a”, “an”, and “the” refer to “one or more” when used in the subject specification, including the claims. Thus, for example, reference to “a reactor” can include a plurality of such reactors, and so forth.

Unless otherwise indicated, all numbers expressing quantities of components, conditions, and so forth used in the specification and claims are to be understood as being modified in all instances by the term “about”. Accordingly, unless indicated to the contrary, the numerical parameters set forth in the instant specification and attached claims are approximations that can vary depending upon the desired properties sought to be obtained by the presently disclosed subject matter.

As used herein, the term “about”, when referring to a value or to an amount of mass, weight, time, volume, concentration, and/or percentage can encompass variations of, in some embodiments +/−20%, in some embodiments +/−10%, in some embodiments +/−5%, in some embodiments +/−1%, in some embodiments +/−0.5%, and in some embodiments +/−0.1%, from the specified amount, as such variations are appropriate in the disclosed packages and methods.

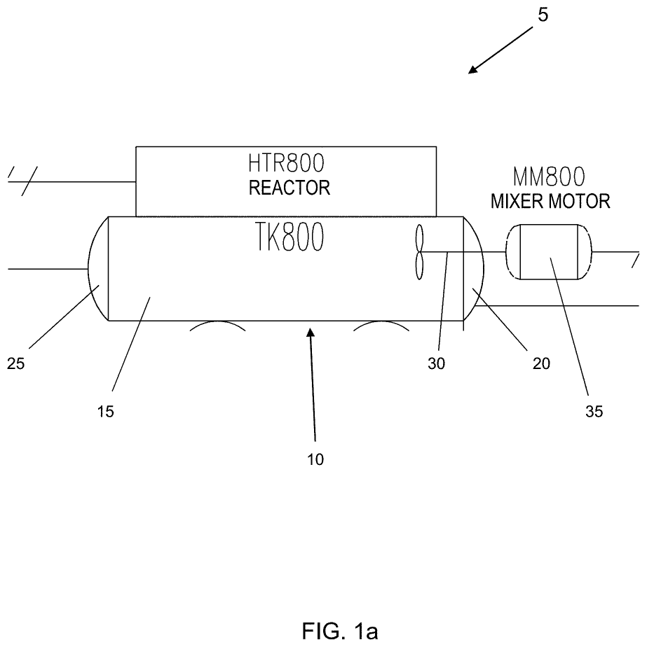

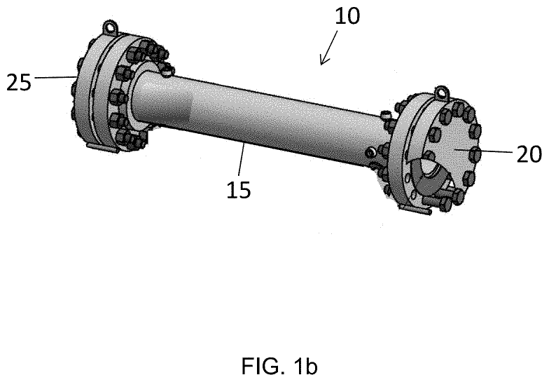

As shown in FIGS. 1a and 1b, the presently disclosed subject matter relates to a horizontal reactor system 5 that can be used for processing various plant materials. For example, the disclosed system can affect partial or total hydrolysis of lignocellulosic materials, generating fermentable carbohydrates as well as paper and pulp products. The disclosed system comprises horizontal reactor vessel 10 comprising body 15 having first end 20 and second end 25. As used herein, the term “horizontal” refers to a direction parallel to the horizontal plane. The term “reactor” as used herein refers to a device for containing or controlling a reaction (e.g., a chemical reaction). Body 15 is illustrated as having a cylindrical shape herein for exemplary purposes only. Thus, it should be appreciated that the body can be configured in any desired shape (e.g., circular, square, rectangular, triangular, abstract, etc.). Reaction vessel 10 is intended to encompass and retain a reaction medium (i.e., biomass slurry) that is to undergo a biological and/or biochemical reaction. Accordingly, it is desirable that vessel 10 be constructed from a material that is inert to the reaction medium. For example, in some embodiments, reaction vessel 10 can be constructed from stainless steel or a suitably lined metallic vessel, or an inert plastic material to prevent introducing unwanted substances into the reaction medium. The reactor comprises first and second hubs 60 positioned at first and second ends 20, 25, respectively, that serve as a sealed end of the reactor. In addition, the hubs cooperate with internal agitator 30 that functions to mix the contents of the reactor, as set forth in more detail below. The term “agitator” as used herein refers to a device that accelerates, stirs, mixes, and/or otherwise energizes flow within a reactor. The agitator is powered by drive 35, which can be a motor, positioned outside the reaction vessel.

The reactor is operated in a horizontal position so that the plane of the travel of the biomass and agitator is horizontal. In some embodiments, the disclosed reactor can process about 1-2 liters of biomass slurry per minute (i.e., 1.0, 1.1, 1.2, 1.3, 1.4, 1.5, 1.6, 1.7, 1.8, 1.9, or 2.0 liters/minute). The reactor can accommodate pressures of up to about 1,550 psi (100 bar) (e.g., 1550, 1500, 1450, 1400, 1350, 1300, 1200, 1100, or 1000 psi or less) and temperatures of up to about 450° F. (232° C.) (e.g., 450, 425, 400, 375, 350, 325, or 300° F. or less). In some embodiments, the reactor can be pressurized using known methods, such as through the use of a nitrogen tank.

Plant materials suitable for use in the disclosed system can comprise plant biomass, agricultural waste, putrescible domestic waste, and intermediate and byproducts thereof. The term “biomass” as used herein refers to any plant-derived matter (woody or non-woody) that is available. For example, biomass can include (but is not limited to) seeds, agricultural crop wastes and residues (such as corn stover, wheat straw, rice straw, sugar cane bagasse, hemp (Cannabis sativa), almond shells, peanut shells, tobacco stalks, and the like), grass crops (such as switch grass, alfalfa, winter rye, and the like), woody crops, wood wastes, and residues (such as trees, softwood or hardwood forest thinnings, barky wastes, branches, pine needles, sawdust, paper and pulp industry residues or waste streams, wood fiber, and the like), food waste, and/or any organic materials. It should be understood that biomass can include agricultural products, non-agricultural products, and all aerial and underground plant parts. Algal and fungal types of biomass can also be included under the term “biomass.” In some embodiments, the biomass can be fresh, partially dried, completely dried, or mixtures thereof (i.e., high moisture, low moisture, and all levels in between). Specific example of biomass suitable for use in the disclosed system can include (but is not limited to) food crops, such as corn, wheat, soybean, cabbage, sugar beets, sugar cane, greens, and the like; non-food crops, such as tobacco, various grasses, bamboo, lavender, algae, Artemisia, hemp, and the like; lumber; chipped wood; agricultural waste (such as corn stover that can be used to produce powdered cellulose, for example); and plant-related industrial waste. In addition, intermediate products made from plants can be used in the disclosed system, such as recycled paper and cardboard, cotton linters, various sacks (i.e., potato sacks, coffee sacks, and the like), barley and/or wheat after beer brewing, paper cups (including coated and uncoated paper cups) can be used.

Biomass slurry can be prepared by combining biomass with a solvent, such as water, NaOH, buffer, and the like. In some embodiments, the ratio of biomass to solvent is at least about 1:7, such as a range of about 1:7 to about 1:20 (e.g., 1:7, 1:8, 1:9, 1:10, 1:11, 1:12, 1:13, 1:14, 1:15, 1:16, 1:17, 1:18, 1:19, or 1:20). The disclosed reactor includes an inlet feed (not shown) that deposits the biomass slurry to be processed into first end 20 of the reactor. In some embodiments, the inlet feed can comprise tubing with diameter of about 1.0 to 0.5 inches (i.e., 1.0, 0.8, 0.75, 0.6, or 0.5 inches). Outlet 40 is positioned at second end 25 of the reactor to allow processed biomass to exit the system. In some embodiments the disclosed system can include a gas outlet pipe for removing gases from the reactor. In some embodiments, the system can include a heat exchanger that can be used to increase the temperature of the reactor contents to a desired level.

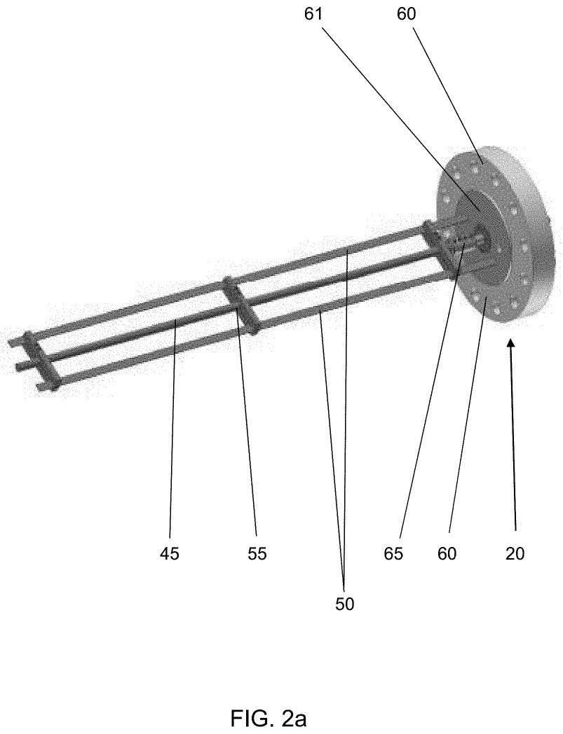

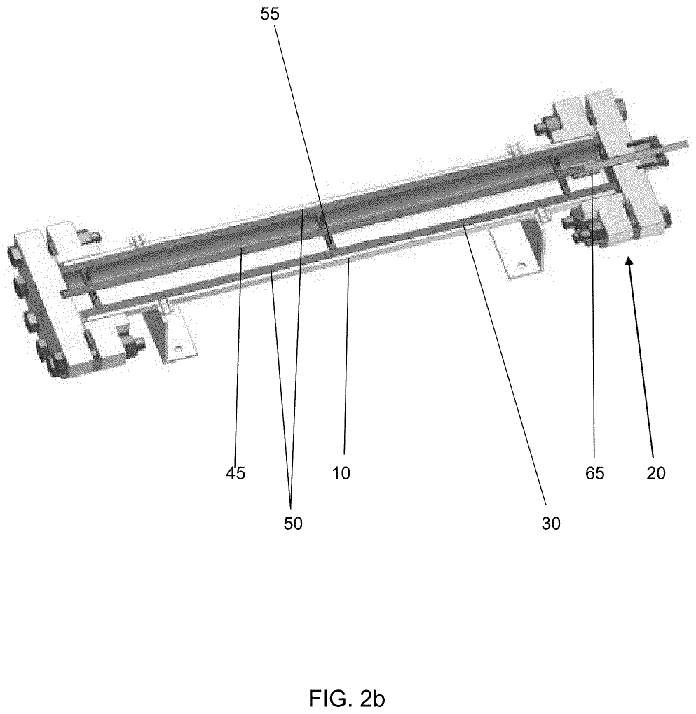

Agitator 30 serves to mix the biomass slurry, break up the biomass, and/or allow the biomass to move towards the second end (discharge end) of the reactor in the longitudinal direction. The agitator speed can be adjustable to allow a user to vary the retention time or production rate through the reactor. FIG. 2a illustrates one embodiment of agitator 30 that can be positioned within horizontal reactor vessel 10, and FIG. 2b illustrates agitator 30 positioned within reactor 10. As shown, the agitator comprises central bore 45 that spans the horizontal length of reactor 10. The central bore comprises a plurality of equidistantly disposed arms 50 that span the distance between the central bore and the inside surface of the reaction vessel. In addition, the segments span the entire length of the reaction vessel to prevent settling of biomass fibers in the corners. Arms 50 can be configured to contact the interior surface of the reactor. Alternatively, the arms can be configured to leave a small gap against the interior reactor wall surface in embodiments where non-contacting travel of the agitator is desired. Accordingly, arms 50 ensure efficient mixing and processing of the biomass within reaction vessel 10. Arms 50 can be connected to central bore 45 by way of one or more connection units 55 that keep the arms in proper position and provide added stability during mixing.

The central bore and arms are operably connected to first and second hubs 60 positioned at first and second ends 20, 25 of the reactor. Specifically, each hub comprises mounting plate 61 within the inner portion of the hub, facing the interior of the reactor vessel. Arms 50 can be connected to the hubs at each end using any method known in the art, such as mechanical closures, welding, adhesives, snap-fit closures, and the like. In addition, central bore 45 can be mounted with or connected to the drive motor via the mounting plate. Any drive motor can that is easily controlled to give a desired speed of rotation can be used. In some embodiments, rotor 65 of the drive motor spans one mounting plate (e.g., the mounting plate at first end 20) such that it contacts the interior of the reaction vessel 5. As shown in FIG. 2a, central bore 45 is connected to the interior portion of the rotor using any method known in the art. Accordingly, the drive motor (via rotor 65) provides rotational motion to the agitator (via central bore 45) in a clockwise or counter clockwise direction about its axis. When the central bore is rotated, arms 50 also are rotated to provide mixing of biomass slurry within the horizontal reaction vessel. Because the arms travel on the interior wall of the reaction vessel, they scrape the biomass material from the wall, whereby the entire biomass volume of the reaction vessel is efficiently mixed and no dead spots are formed. Thus, the reaction medium (biomass slurry) is mixed when the torque of the drive motor is transmitted to the central bore and segments of the agitator. The motor can be actuated by a command signal from a control unit (not shown) that directs the agitator to rotate. By controlling the agitation conditions in the reaction vessel, it is possible to employ various states of speeds of agitation to assist the reaction process. For example, in some embodiments, the agitator can be adjustable to about 15-120 revolutions per minute (RPM), such as at least about (or no more than about) 15, 20, 25, 30, 35, 40, 45, 50, 55, 60, 65, 70, 75, 80, 85, 90, 95, 100, 105, 110, 115, or 120 RPM.

Arms 50 and central bore 45 can be formed as flat rectangular units as shown in FIGS. 2a and 2b. In some embodiments, the arms comprise a straight edge to scrape the interior surface of the reaction vessel so that all material around the interior surface of the vessel can be mixed. However, it should be appreciated that the central bore and agitator arms can be configured in any shape, so long as rotation promotes mixing of the biomass slurry (i.e., wedge-shaped, fork-shaped, plates). In some embodiments, the central bore and/or arms can be freely adjusted to any form suitable for the desired mixing purposes (i.e., the arms can be changed to suit different biomass materials or to allow replacement when worn).

Arms 50, central bore 45, and/or agitator 30 can be constructed from any non-reactive material known and used in the art. For example, in some embodiments, the cited elements can be constructed from polymeric material, metal, ceramic material, and the like.

In some embodiments, the arms, central bore, and/or agitator can be molded as a unitary assembly. Alternatively, the agitator can be constructed as a series of individual units that are assembled together using methods well known in the art.

In some embodiments, reactor 10 comprises connectors for liquid and gas inputs, a viewing glass for monitoring the progress of the reactions in the vessel, and/or an area for instrumentation (such as temperature, pH probes), and a sample port. All the above are optional and used as need.

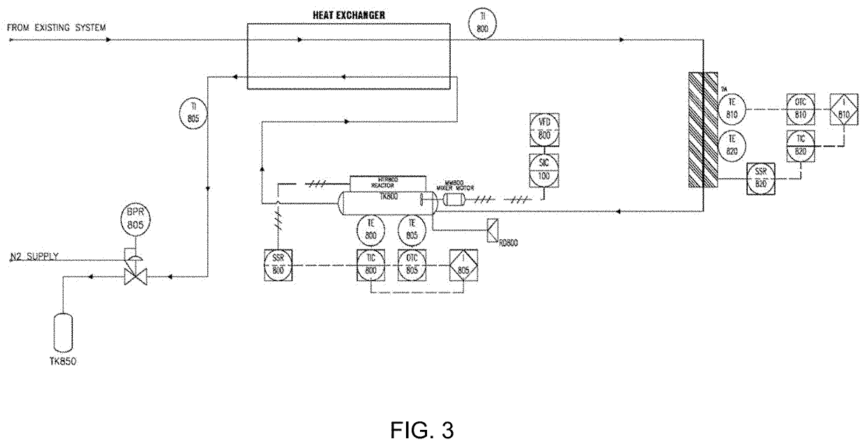

FIG. 3 illustrates one embodiment of processing system 70 that can include horizontal reactor system 5. Particularly, in some embodiments, biomass from an existing system (i.e., mechanical pre-treatment, main tank, feeding pump, and high-pressure pump) can travel through heat exchanger 75 to allow the biomass slurry to reach a desired temperature. The biomass slurry then travels through horizontal reactor system 5 where the biomass is processed. Processed biomass then exists the horizontal reactor and travels through heat exchanger 75 to set the biomass slurry to a desired temperature.

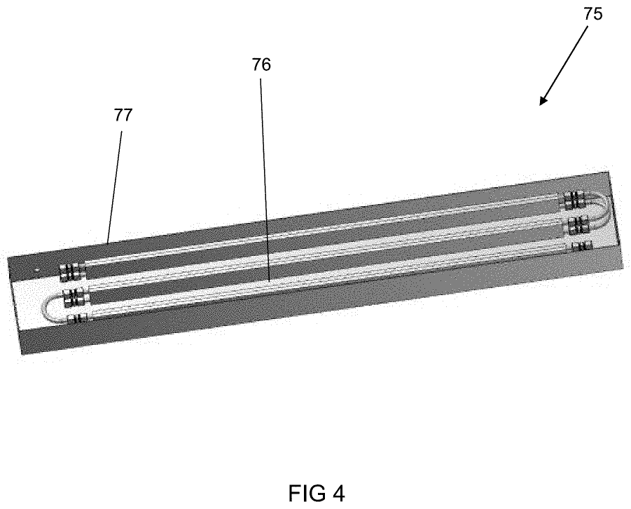

One embodiment of heat exchanger 75 is shown in FIG. 4. Particularly, heat exchanger 75 can be used to heat the biomass slurry to a desired temperature. In some embodiments the heat exchanger can comprise a thermostat and one or more heating elements 76 contained in housing 77. In some embodiments, the heating elements comprise metal tube elements housing heating or cooled liquid (e.g., water). Advantageously, the heat exchanger can enable recovery of the majority of the heat from the subcritical water to improve the overall efficiency of the system.

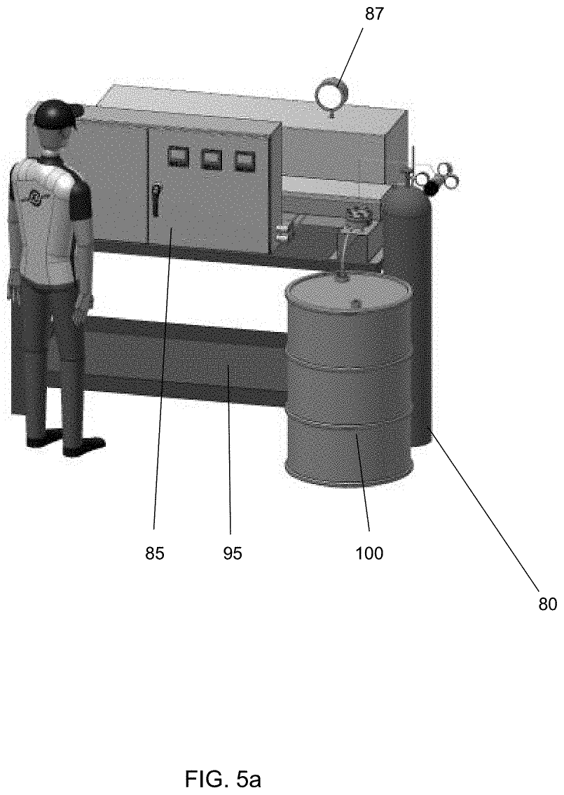

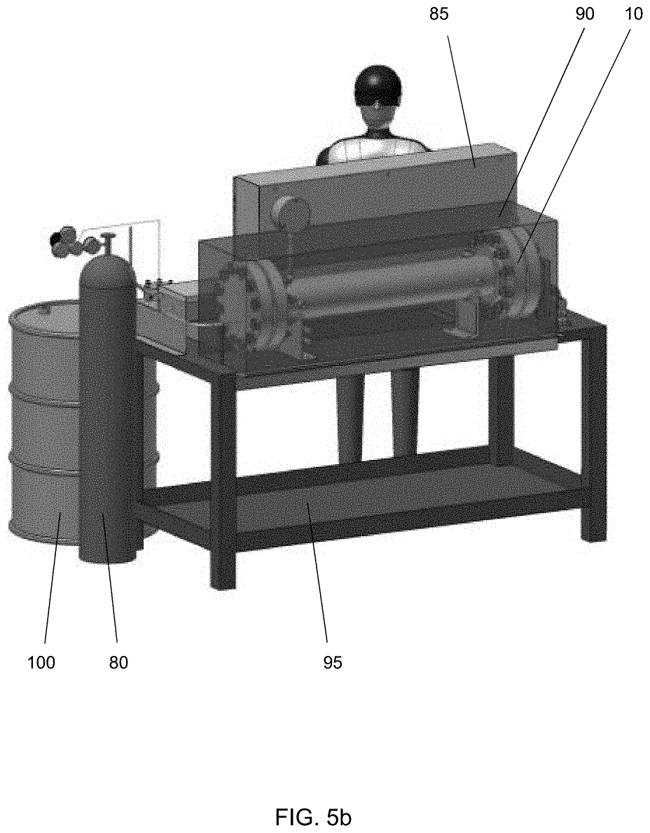

FIGS. 5a and 5b illustrate front and rear views of one embodiment of the disclosed system. Particularly, the system comprises pressurized gas tank 80 that functions to control one or more valves in the system. In some embodiments, tank 80 comprises nitrogen. The system also comprises control panel 85 that allows the user to monitor and regulate the system as it is running (i.e., temperature, pressure, and the like). In some embodiments, the system comprises one or more monitors 87 that can monitor temperature and/or pressure. The system further comprises horizontal reactor 10 positioned within protective housing 90. In some embodiments, the system can rest on a support surface (such as table 95) to make operation and monitoring easier for the user. The final reaction product (i.e., sugar in some embodiments) can be collected in receptacle 100.

In some embodiments, the disclosed horizontal reactor comprises prefabricated modular elements with programmed automatic or manual operation, such that it can be easily moved in and assembled on site without undergoing expensive and time-consuming system elements stoppage. Thus, the disclosed reactor can include a combination of interchangeable and replaceable modules and sections that allow flexible operation and switching from one type of feedstock to another. In some embodiments, the disclosed reactor can be transported to a biomass capture facility or site.

It should also be appreciated that the size of the disclosed system can be scaled up or down, depending on the size constraints by the user, biomass to be processed, and the like. For example, in some embodiments, the disclosed system can be about 6 feet long, 3 feet wide, and 7 feet tall. In some embodiments, the reactor works via electric connections. Further, in some embodiments the reactor vessel can have a volume of about 20 liters up to multi-ton volumes. It should be appreciated that reactor size is generally based on the required residence time of the biomass and the flow rate.

### EXAMPLES

The following Examples have been included to provide guidance to one of ordinary skill in the art for practicing representative embodiments of the presently disclosed subject matter. In light of the present disclosure and the general level of skill in the art, those of skill can appreciate that the following Examples are intended to be exemplary only and that numerous changes, modifications, and alterations can be employed without departing from the scope of the presently disclosed subject matter.

### Example 1

### Hydrothermal Horizontal Reactor Processing of Various Feedstock Materials

Several plant materials (wheat straw, tobacco, corn stover, bagasse, pine, and eucalyptus) were processed in a continuous pilot scale hydrothermal reactor of the type disclosed herein to demonstrate the efficiency of delignification. The reactor reaction pressure ranged from 150-1,100 psi, temperature ranged from 150-300° C., reaction time ranged from 5-35 minutes, % NaOH ranged from 0.5-10%, and flow rate ranged from 1-2 liters of biomass per minute. The percent lignin was measured in accordance with TAPPI T236, incorporated herein by reference. The results are shown in Table 1 below.

TABLE 1
Hydrothermal Horizontal Reactor Delignification Efficiency
for Processing Various Biomass Materials

| | | Lignin % | |
|---|---|---|---|
| | Initial | in Produced | Lignin |
| Biomass | Lignin % | Pulp | Reduction (%) |
| Wheat Straw | 18.7 | 1.3 | 91.1 |
| Tobacco | 19.0 | 1.3 | 93.5 |
| whole plant | | | |
| Corn stover | 15.0 | 1.3 | 91.7 |
| Bagasse | 22.9 | 1.0 | 95.5 |
| Pine | 30.0 | 1.0 | 96.7 |
| Eucalyptus | 27.0 | 2.3 | 91.7 |

### Example 2

### Hydrothermal Horizontal Reactor Processing of Wheat Straw

Wheat straw was used as a feedstock for demonstrating pulp production mass balance through a continuous pilot scale hydrothermal horizontal reactor and post-processing products (such as dissolving pulp). The results are shown in Tables 2 and 3, below. In Table 2, data was recorded for a duration of 5 hours of processing, and pulp was generated without a refining process using an initial NaOH concentration of 0.5-5%. In Table 3, the initial unbleached wheat straw pulp was produced from the continuous pilot scale hydrothermal horizontal reactor, and the bleaching process was based on a totally chlorine-free (TCF) bleaching sequence. As shown in Table 3, the produced dissolving pulp had a high cellulose content (>90%), adjusted viscosity (degree of polymerization), low extractives content, and low adjusted hemicellulose content.

TABLE 2
Amount of Wheat Straw Pulp Produced using a Continuous
Pilot Scale Hydrothermal Horizontal Reactor

| Feedstock | Output | Equivalent | | | Recovered |
|---|---|---|---|---|---|
| Input | Wet | Dry | NaOH | | Sugars* |
| (dry wt, | Pulp | Pulp | Consumption | Pulp Yield | Yield |
| kg) | (kg) | (kg) | (%) | (%) | (%) |
| 16.0 | 114.5 | 12.3 | 27.0 | 77.1 | 11.4 |

“*Recovered Sugars” represents the fermentable sugars and other carbohydrates.

TABLE 3
Properties of Bleached Wheat Straw Dissolving Pulp

| | Wheat Straw | |
|---|---|---|
| Analysis | Dissolving Pulp | Unit |
| Intrinsic Viscosity | 4.28 | dLg |
| S18 | 5.56 | % |
| Acetone Extractives | 0.077 | % |
| Alpha Cellulose | 91.0 | % |
| Ash @525° C. | 0.53 | % |
| Brightness | 80.3 | % |
| Fock Reactivity | 70.5 | % |
| (% Dissolved Cellulose) | | |

### Example 3

### Analysis of Carbohydrate Components in the Black Liquor of Pulp Production

The carbohydrate components in the black liquor from the pilot scale reactor through the pulp production process were analysed. The flow rate of the generated liquor was about 0.8-1.2 L/min. Tested feedstocks include bagasse and wheat straw. The sugar analysis was performed using HPLC (flowrate: 0.6 mL/min; temperature: 85° C.; syringe filters: 0.22 micron PVDF). The samples were hydrolyzed as described by NREL sulfuric acid protocols without any dilution and filtered for HPLC and other analyses.

TABLE 4
Carbohydrate Components in Black Liquor of
Pulp Production from Pilot Scale Reactor

| | |
|---|---|
| Black Liquor | |
| Carbohydrate | Feedstocks |

| Components (g/L) | Bagasse | Wheat Straw |
|---|---|---|
| Glucose | 0.519 ± 0.013 | 0.917 ± 0.008 |
| Xylose | 2.174 ± 0.009 | 2.514 ± 0.042 |
| Galactose | 0.280 ± 0.012 | 0.421 ± 0.031 |
| Arabinose | 0.229 ± 0.040 | 0.802 ± 0.015 |
| Mannose | 0.255 ± 0.048 | 0.277 ± 0.053 |

What is claimed is:

1. A horizontal bioreactor apparatus comprising:
a horizontal reaction vessel comprising an interior and first and second ends;
a first hub positioned at the first end of the vessel;
a second hub positioned at the second end of the vessel; and
an agitator positioned within the interior of the vessel, the agitator comprising:
a central rotating bore operably connected to the first and second hubs; and
one or more arms connected to the central rotating bore and operably connected to the first and second hubs.

2. The apparatus of claim 1, wherein the horizontal reaction vessel is constructed from stainless steel or a polymeric material.

3. The apparatus of claim 1, wherein the central rotating bore is coupled to an external drive device via the first or second hub.

4. The apparatus of claim 3, wherein the external drive device is a motor.

5. The apparatus of claim 1, wherein the horizontal reaction vessel is capable of processing about 1-2 liters of biomass slurry per minute.

6. The apparatus of claim 1, wherein the horizontal reaction vessel can accommodate pressures of up to about 1,550 psi.

7. The apparatus of claim 1, wherein the horizontal reaction vessel can accommodate temperatures of up to about 450° F.

8. The apparatus of claim 1, wherein the horizontal reaction vessel r is pressurized through the use of a gas tank.

9. The apparatus of claim 8, wherein the gas is nitrogen.

10. The apparatus of claim 1, wherein the arms contact the interior surface of the reactor.

11. The apparatus of claim 1, further comprising a viewing glass for monitoring progress of the reactions in the vessel.

12. The apparatus of claim 1, further comprising a sample port.

13. A horizontal bioreactor system comprising:
a mechanical pre-treatment module;
a bioreactor module comprising:
a horizontal reaction vessel comprising an interior and first and second ends;
a first hub positioned at the first end of the vessel;
a second hub positioned at the second end of the vessel;
an agitator positioned within the interior of the vessel, the agitator comprising:
a central rotating bore operably connected to the first and second hubs;
one or more arms connected to the central rotating bore and operably connected to the first and second hubs; and
a collection module.

14. The system of claim 13, wherein the mechanical pre-treatment module comprises a hammer mill.

15. The system of claim 13, further comprising a heating module comprising a heat exchanger.

16. The heat exchanger of claim 15, comprising metal tube elements housing heated or cooled liquid.

17. A method of processing biomass to produce fermentable carbohydrates, paper and pulp products, or both, the method comprising:
preparing a biomass slurry comprising a biomass to be treated;
introducing the biomass slurry to a horizontal bioreactor apparatus, wherein the horizontal bioreactor apparatus comprises:
a horizontal reaction vessel comprising an interior and first and second ends;
a first hub positioned at the first end of the vessel;
a second hub positioned at the second end of the vessel;
an agitator positioned within the interior of the vessel, the agitator comprising:
a central rotating bore operably connected to the first and second hubs;
one or more arms connected to the central rotating bore and operably connected to the first and second hubs;
allowing the horizontal bioreactor apparatus to proceed for a desired amount of time; and
collecting end products of fermentable carbohydrates, paper and pulp products, or both.

18. The method of claim 17, wherein the biomass is selected from corn, wheat, soybean, cabbage, sugar beet, sugar cane, greens, tobacco, bamboo, lavender, algae, Artemisia, cotton linter, hemp, lumber, chipped wood, corn stover, potato sacks, coffee sacks, paper cups, and combinations thereof.

19. The method of claim 17, wherein the biomass slurry comprises biomass and a solvent selected from water, NaOH, buffer, and combinations thereof.

20. The method of claim 17, wherein the ratio of biomass to solvent is about 1:7 to 1:20.
